(19) United States
(12) Patent Application Publication
(10) Pub. No.: US 2007/0203396 A1
(43) Pub. Date: Aug. 30, 2007

(54) Endoscopic Tool

(76) Inventors: John G. McCutcheon, Menlo Park, CA (US); Antony J. Fields, San Francisco, CA (US); Ronald R. Hundertmark, San Mateo, CA (US); Michael Hendricksen, Redwood City, CA (US); Michael Barrett, Campbell, CA (US); Alan R. Rapacki, Redwood City, CA (US); Garrett Schwab, Oakland, CA (US)

Correspondence Address: FISH & RICHARDSON, PC, P.O. BOX 1022, MINNEAPOLIS, MN, 55440-1022, US

(21) Appl. No.: 11/679,635

(22) Filed: Feb. 27, 2007

Related U.S. Application Data
(60) Provisional application No. 60/777,933, filed on Feb. 28, 2006.

(51) Int. Cl. A61B 1/06

(52) U.S. Cl. 600/173; 600/170

(57) ABSTRACT

Delivery system including endoscopes are described. In an embodiment, a delivery system includes a flexible, elongate endoscope extending along a longitudinal axis when the endoscope is straightened and a first lens located on the distal region. The first lens faces a direction transverse to the longitudinal axis. The first lens permits the endoscope to provide an image of anatomical structures that offset from the axis of the endoscope.

### REFERENCE TO PRIORITY DOCUMENT

This application claims priority of U.S. Provisional Patent Application Ser. No. 60/777,933, filed Feb. 28, 2006. Priority of the aforementioned filing date is hereby claimed and the disclosure of the Provisional Patent Application is hereby incorporated by reference in its entirety.

### BACKGROUND

The present disclosure relates generally to methods and devices for use in performing medical procedures including delivery devices and procedures for the lungs.

The flexible endoscope has existed in its current form for quite some time. An image is gathered at the distal tip of the endoscope and transmitted to the proximal end of the endoscope either through a bundle of coherent optical fibers, or through a video camera located at the distal tip. The image is transmitted electronically through the endoscope and to an image processor and displayed on a monitor. The flexible endoscope is available with a biopsy or “working” channel that runs the length of the endoscope and allows tools to be inserted into the channel at the handle and through the endoscope to the tip, to allow the suctioning of secretions, etc. The tip of the endoscope is deflectable in one or more directions to allow the device to be steered through the body lumen during insertion.

One type of endoscope is the bronchoscope, which is specifically designed to inspect and treat the lungs. A flexible fiberoptic bronchoscope 120 is shown in FIG. 1 and includes an eyepiece 140, a handle 125, a working channel entrance 135, a flexible shaft 130, and a distal tip 145. The distal tip of the bronchoscope is shown in FIG. 2, and includes the distal opening of the working channel 710, one or more illumination sources 730, and the image collector 720, such as a video camera or fiberoptic bundle end for example. In the last few years, a number of new interventional bronchoscopy procedures have emerged that utilize flexible bronchoscopes in diagnostic or treatment procedures.

Such procedures include the implantation of bronchial flow control devices such as blockers, one-way valves and two-way valves, bronchial by-pass procedures, tracheobronchial stent placement procedures, transbronchial needle biopsy procedures, bronchoscopic treatment of air leaks, laser therapy, cryotherapy, photodynamic therapy, etc. FIG. 3 illustrates a bronchial flow control device 105 being implanted in a bronchial lumen of the lungs with a delivery catheter 110 placed through the working channel of a flexible bronchoscope 120.

There are a number of ways in which either the flexible bronchoscope could be modified or improved, or ancillary devices could be designed, modified or improved, to assist in the performance of these and other pulmonary procedures. Several of these devices and improvements are described herein.

### SUMMARY

Several embodiments of endoscopic devices and methods of use are described herein. In one aspect, there is disclosed a delivery system for insertion into a lung. The delivery system includes a flexible, elongate endoscope extending along a longitudinal axis when the endoscope is straightened and a first lens located on the distal region, wherein the first lens faces a direction transverse to the longitudinal axis. The first lens permits the endoscope to provide an image of anatomical structures that offset from the axis of the endoscope. For example, the walls of body lumens can be viewed.

In another aspect, there is disclosed a delivery system for insertion into a lung. The delivery system includes a flexible, elongate endoscope extending along a longitudinal axis when the endoscope is straightened. The system further includes a depth sensor adapted to be coupled to the endoscope and to a landmark relative to the patient's body. The depth sensor is adapted to provide an electronic signal that indicates the distance between the distal tip of the endoscope and the entrance location.

In another aspect, there is disclosed a delivery system for insertion into a lung. The delivery system comprises a flexible, elongate endoscope extending along a longitudinal axis when the endoscope is straightened; a first lens facing a direction normal to the longitudinal axis; and a lumen size measurement device coupled to a distal region of the endoscope. The lumen size measurement device is adapted to provide an indication of the size of a bronchial lumen in which the distal region of the endoscope is positioned.

In another aspect, there is disclosed a delivery system for insertion into a lung. The delivery system comprises an elongate body having a proximal end and a distal region. The elongate body is adapted to be inserted into the lung via a patient's mouth. The system further comprises a balloon attached to the distal region. The balloon is adapted to be inflated in an asymmetrical manner such that the balloon expands in only one direction relative to a longitudinal axis of elongate body.

In another aspect, there is disclosed a delivery system for insertion into a lung. The delivery system comprises a flexible endoscope adapted to be inserted into the lung and an orientation indicator coupled to the flexible endoscope. The orientation indicator is adapted to provide an indication as to the direction to an anatomical structure of a patient in which the endoscope is inserted regardless of the orientation of the endoscope within the patient.

Other features and advantages should be apparent from the following description of various embodiments, which illustrate, by way of example, the principles of the invention.

### BRIEF DESCRIPTION OF THE DRAWINGS

FIG. 1 shows a first embodiment of a bronchoscope.

FIG. 2 shows a distal region of the bronchoscope.

FIG. 2A shows an alternate embodiment of a bronchoscope.

FIG. 3 shows a flow control device being deployed from a delivery catheter placed through the working channel of a bronchoscope and into a target bronchial lumen.

FIG. 4 shows a distal region of an alternate embodiment of a bronchoscope.

FIG. 5 shows a bronchoscope having an inflatable balloon.

FIG. 6 shows another embodiment of a bronchoscope.

FIG. 7 shows a cross-sectional view of a portion of the bronchoscope of FIG. 6.

FIG. 8 shows the bronchoscope of FIG. 6 deployed in a bronchial tree.

FIG. 9 shows an end-view of the distal end of a bronchoscope.

FIG. 10 shows an end-view of the distal end of another embodiment of a bronchoscope.

FIG. 11 shows an end-view of the distal end of another embodiment of a bronchoscope.

FIG. 12 shows another embodiment of a bronchoscope.

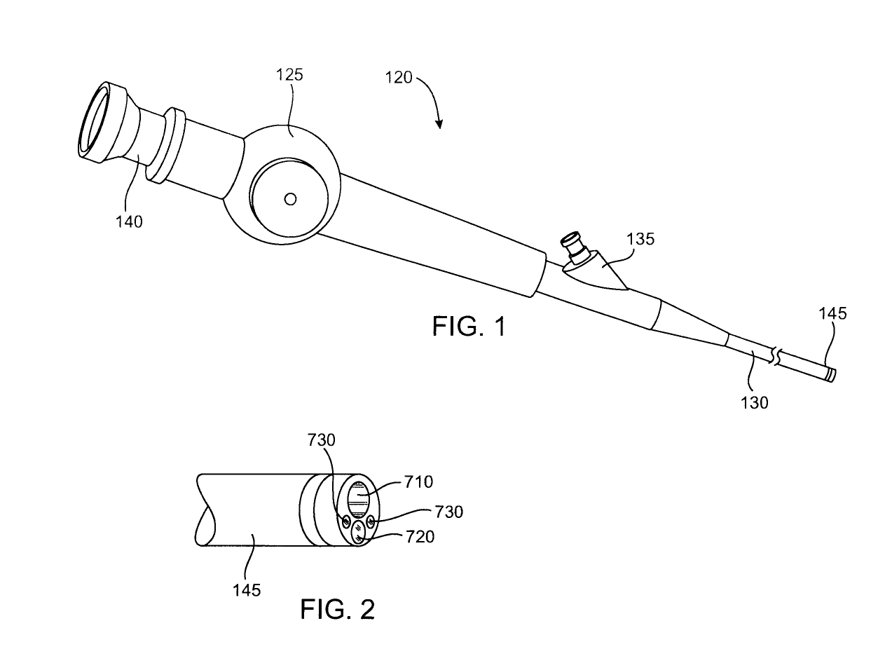

### DETAILED DESCRIPTION

Improvements to the imaging capabilities and other capabilities of an endoscope are described herein. For the purposes of this disclosure, the term endoscope will be used to mean any endoscope used to view the inside of the body, either through an incision or orifice. This includes flexible and rigid bronchoscope, gastroscopes, ureterscopes, etc. The disclosed devices are sometimes described herein in the context of a bronchoscope. However, it should be appreciated that the device features described herein can sometimes be used with any type of endoscope.

Endoscope with Zoom Lens

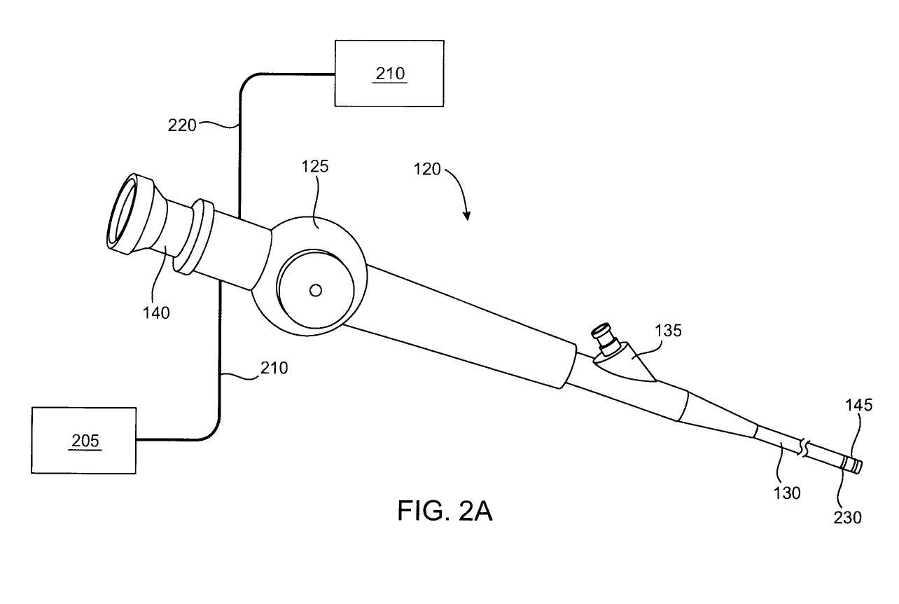

It would be advantageous if the image returned by the endoscope could be zoomed so that a small detail in the field of view could be expanded to allow the image to be viewed in greater detail. There are also situations where it would be advantageous to zoom back to allow a larger field of view to be seen in the image monitor. In one embodiment, a mechanical zoom lens is located at the distal tip or distal region of the endoscope to allow the field of view to be zoomed. For example, the lens could be mounted within the distal tip 145 of the endoscope shown in FIG. 2 such as at or within the location 720. The endoscope includes an actuator that is located at a distal region of the endoscope for adjusting the lens. The lens could be adjusted manually at the proximal tip (such as the handle 125) of the endoscope, and the endoscope reinserted into the patient.

In another embodiment, a remote adjustment is located at the endoscope handle or elsewhere. The remote adjustment is actuated to adjust the zoom value of the lens mechanically, electrically or otherwise. A mechanical zoom can be used wherein the lens element or elements at the tip of the endoscope are moved relative to each other in order to change the amount of zoom. In another embodiment, the endoscope is coupled to a digital post processor 205 such that the image zoom is achieved through digital post-processing of the video image, as represented schematically in FIG. 2A. This is achieved by electrically enlarging a selected portion of the image that is projected in the image display.

In another embodiment, the lens at the tip of the endoscope is replaceable by the user, and different lenses such as fish eye lenses, telephoto lenses, wide angle lenses, etc. are removably attached to the tip of the endoscope to alter the field of view of the image. For example, the distal region shown in FIG. 2 could comprise a removably mounted module that removably couples to the endoscope. The various lens modules can be attached with screw threads, a bayonet lock, or any other appropriate attachment method. The lens modules have appropriate working channel and illumination openings if the module encompassed the working channel and/or illumination outputs on the tip of the endoscope. If the image is captured by a CCD camera or other electronic image collector, the necessary electronic connections are disconnected automatically when the lens module is removed, and reconnected when it is attached.

Endoscope with Side View Lens

In another embodiment, a endoscope is adapted to allow an image to be collected that is facing the bronchial lumen wall rather than facing down the length of the bronchial lumen. This allows the user to examine the bronchial wall in detail and at an angle that is normal to the lumen wall (and normal to the centerline of the endoscope) or at any other angle relative to the centerline of the endoscope. In one embodiment, a lens with a right angle prism is located on the endoscope, such as at the distal tip or distal region of the endoscope to allow illumination and viewing of the sidewall. In another embodiment, the endoscope has a CCD or other camera mounted in the tip of the endoscope.

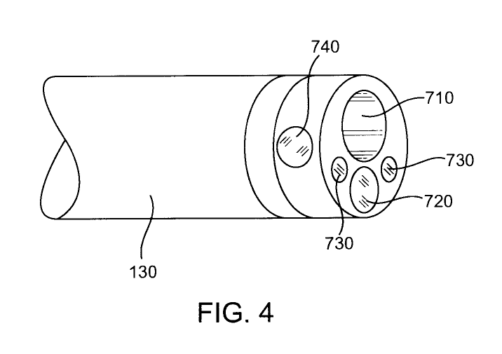

As mentioned, the endoscope can have one or more changeable tips that contain the camera, and connect electrically with the shaft of the endoscope when attached to the distal tip of the endoscope in order to carry the video signal back to the image processor and/or display. One tip has a standard forward-looking camera, while others have a camera aimed directly sideways, 90 degrees to the centerline of the endoscope, while still others have a camera focused at any angle in between or greater than 90 degrees. In an alternative embodiment, as shown in FIG. 4, the distal tip of the endoscope 145 has two or more image collectors such as a camera or fiberoptic bundle. The user can select whether to view the image from one image collector, the other image collector, or both in the image monitor at any given time. For example, as shown in FIG. 4, a first image collector 720 returns an image of the standard endoscope view facing in the distal direction parallel to the centerline of the endoscope. The endoscope also includes a second image collector 740 on a side of the endoscope. The side facing image collector 740 can face the bronchial lumen wall for example.

Endoscope with Fish-Eye Correction

The standard tip lens set-up on a flexible endoscope gives the user a “fish-eye” or very wide angle view of the airway under examination. This allows the distal airway to be imaged, along with some of the bronchial lumen wall surrounding the endoscope tip. The disadvantage of this is that the view of the airway is distorted, and the viewed objects appear farther away from the lens than they really are. In another embodiment, the endoscope is adapted to capture an image digitally. In this regard, a camera or other image collector is disposed at the distal tip of the endoscope, or at the proximal end of the endoscope such as at the handle. The digital image in a normal state may be a somewhat distorted wide-angle image. The digital image is digitally altered (such as by using the processor 205) to flatten the image and appear as if the image were taken through a non-fisheye lens. This alteration of the image can be accomplished before the image is projected on the image monitor or viewing screen. The user has the option of viewing the image in the unprocessed fish-eye mode, or in the processed flattened mode, or in any other processed version of the original image.

Wireless Endoscope

Existing flexible endoscopes, rigid endoscopes and other endoscopes are connected to one or more instruments by either wire bundles or by fiberoptic bundles. It is necessary to provide illumination to the tip of the endoscope in order to capture a good image, and this is typically done through the use of one or more fiberoptic bundles that run from the tip of the endoscope through the endoscope and out through the endoscope handle and through an external fiberoptic cable to a light source. There are also one or more electrical cables connecting the endoscope camera to an external processing unit. All of these cables add to the weight of the endoscope, and inhibit its mobility.

It would be very advantageous to eliminate the cable that connects the endoscope to instruments in the procedure room. In one embodiment, the video signal from the endoscope is transmitted wirelessly to the processing unit in the procedure room. FIG. 2A schematically shows the endoscope coupled to the processor 205 and/or an image display 210 via connections 215 and 220, respectively. The connections 215 and 220 can be either wired or wireless connections. The wireless connections can be done through radio frequency (RF) connection, infrared connection (IR) or other wireless data transmission method. In one embodiment, batteries (rechargeable or otherwise) are coupled to the endoscope as a power source for this wireless connection.

In another embodiment, the light source is internal to the endoscope and is powered by batteries in the endoscope handle. The light source could be in the handle with the light transmitted to the tip of the endoscope by a fiber optic bundle or bundles. Alternately, the light source can be in the tip of the endoscope in the form of high power light emitting diodes or other compact light sources. In one embodiment, the power source for the endoscope are rechargeable batteries, and these batteries are recharged by removing a battery pack from the endoscope and placing it in a battery charging station that is connected to AC power. In another embodiment, the batteries are charged by plugging a charging cable into the endoscope. In yet another embodiment, the batteries are charged automatically when the endoscope is hung on a storage bracket. Electrical connections to a battery charger are automatically connected when the endoscope is hung on the storage bracket. This allows the batteries to be charged whenever the endoscope is not in use.

Lumen Size Measurement

With many of the procedures that are performed with endoscopes, it is necessary to measure certain dimensions of the body cavity that is being examined and treated. For example, it is often necessary to measure the diameter and/or length of a bronchial lumen when performing a pulmonary procedure with a bronchoscope. If a tracheobronchial stent is being implanted, it is necessary to know the diameter and length of the lumen being stented in order to select the correct size of implant. Currently this is done with various methods and measuring tools including measuring the diameter with an inflated balloon catheter, measuring the diameter by comparing the known endoscope diameter to the lumen diameter, measuring the length with a catheter placed through the working channel that has length marks placed on the shaft, etc. These measuring procedures could be greatly improved by incorporating a measuring device into the endoscope. There are a number of different ways in which this could be done. Some examples are now described:

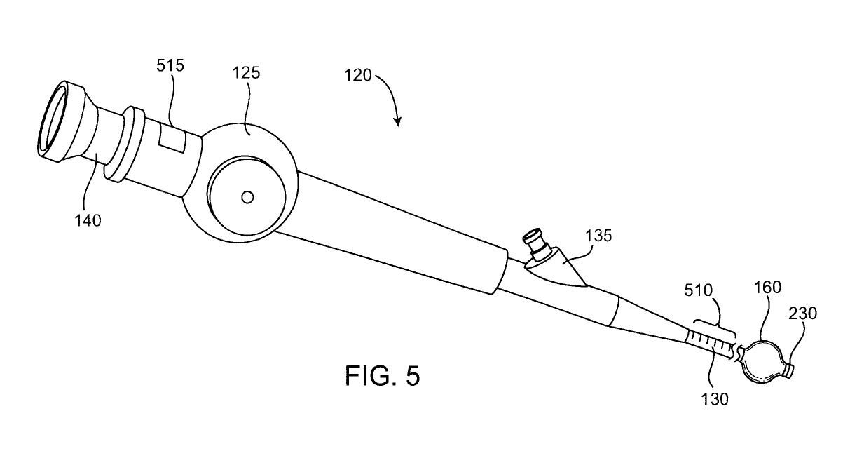

(1) In one embodiment, as shown in FIG. 5, an inflatable balloon 160 is incorporated into the distal tip of the endoscope 120. The inflatable balloon is coupled to a source of fluid that permits the balloon to be inflated and enlarged in size. In use, the balloon is slowly inflated while moving the endoscope distally and proximally through the bronchial lumen until the balloon contacts the lumen walls, and restricts further distal and proximal movement of the endoscope. The amount of air or fluid injected into the balloon is noted, the balloon deflated, the endoscope removed, the balloon re-inflated with the same quantity of fluid, and finally the outer diameter of the balloon, corresponding to the inner diameter of the lumen being measured, is itself measured. Alternately, the balloon diameter at different injection volumes is measured, and a calibration curve derived that allows the lumen diameter to be estimated given the amount of fluid injected into the balloon.

(2) In an alternative embodiment, an ultrasound sensor may be used to measure the lumen diameter. This is commonly done in the vasculature with a system known as intravascular ultrasound or IVUS. With IVUS, an ultrasound sensor mounted on the distal tip of a catheter is inserted into the vasculature and translated to the area of interest, typically an occlusion or stenosis of the vessel. The output of the sensor is displayed on a viewing screen and is a circular cross sectional image of the vessel that is perpendicular to the centerline of the lumen. The image is shaded to delineate areas of different density in the viewed tissue.

An ultrasonic sensor 230 (schematically shown in FIGS. 2A and 5) is mounted on or in the distal tip of the endoscope and is coupled to the bronchial lumen wall by inflating a fluid filled balloon or other flexible bladder mounted at the tip of the endoscope. Alternately, an ultrasound sensor is mounted on a distal tip of a catheter that is deployed through the endoscope. This permits a cross sectional density map of the bronchial lumen to be displayed. A fluid filled balloon or bladder can be used to permit transport of the ultrasound signal. Thus, a coupling medium in the form of a fluid (typically saline) filled bladder is used. The sensor is calibrated prior to the procedure so that an accurate estimate of the lumen diameter may be determined from the resultant image. The displayed image is then measured to determine the diameter of the bronchial lumen at the current location of the ultrasound sensor. The endoscope may be moved proximally or distally in order to measure the lumen diameter in multiple locations. Other non-contact sensing technologies could also be employed to measure lumen diameter or length such as laser range finders, ultrasonic range finders, etc.

Endoscope Location and Orientation Determination

During insertion of an endoscope into the body, and after rotation and movement of the endoscope during an examination or procedure, it is common for the operator to get confused and to forget where the tip of the endoscope is located and what rotational orientation the endoscope is in. In addition, when a complex anatomical structure is examined, such as the bronchial tree of the lungs, it is quite easy for the operator to forget which lung segment or lobe the tip of the endoscope is in. In these situations, the operator typically will withdraw the endoscope until a major landmark such as the main carina is visible, and then reinsert the endoscope. It would be very helpful to provide the user with some aids in determining the current location and/or orientation of the distal tip of the endoscope. There are now described various embodiments of endoscopes that include position and/or orientation aids.

Endoscope with Orientation Sensor

It would be very helpful to the operator if an indicator, such as a marker or arrow, is displayed in the display 210 that is projecting the image from the tip of the endoscope, wherein the indicator indicates the direction to a particular portion of the anatomy of the patient under examination, regardless of the orientation of the endoscope. In the example of a endoscope, an arrow can be displayed that always points in the direction of the spine of the patient being examined. When the endoscope is rotated, the arrow moves on the display screen to always point in the direction of the spine of the patient, thus giving the operator a real-time update on the orientation of the endoscope.

This can be accomplished in a number of ways. In one embodiment, a rotation sensor is located on the handle end of the endoscope wherein the sensor returns the angle that the endoscope is rotated relative to a landmark on the patient, such as a bite block or endotracheal tube placed in the patient's mouth, or to any other device that is fixed relative to the patient's body. The sensor can be a device that requires contact between the endoscope and the device fixed to the patient's body such as digital encoder, rotational resistor or any other device that detects rotation. Alternately, the sensor can be a non-contact device that can sense the relative rotation between the endoscope and a device fixed to the patient's body. This sensor can be inductive, optical, magnetic, or any other technology that allows non-contact rotation sensing between the endoscope and a fixed device.

The endoscope orientation is then calibrated by entering a setup mode whereby the endoscope is rotated until an indicator visible in the display and fixed to the endoscope is pointing down towards the patient's back at a location in an airway, such as the trachea, where the down or posterior direction is obvious. The orientation is then set as the “down” direction, and when the endoscope is rotated relative to the bite block or ET tube, the down arrow projected in the monitor rotates with the image so that it continues to point in the posterior direction. Alternately, the indicator can be set up to always point towards the chest of the patient, or to any other direction.

Alternately, a directional radio frequency (RF) sensor is mounted in the tip of the endoscope, and an RF emitter pad is placed under the back of the patient. The strength, direction, or a combination of strength and direction of the signal determines the orientation of the sensor and thus the orientation of the endoscope. Of course, there are numerous other methods of sensing endoscope orientation not mentioned here.

Insertion Depth Sensor

On many occasions, the physician would like to remove the endoscope from the patient in order to perform an operation, such as cleaning the tip of the endoscope, and then reinserting the endoscope back in the patient to the same location. It can often be difficult to return the tip of the endoscope to the same location based on the clinician's memory of the appearance of the target location. This process could be aided by marking the outside of the endoscope shaft with depth marks 510, such as shown in FIG. 5. The clinician merely notes the depth of the endoscope prior to removing it. Thus, an embodiment of the endoscope includes a series of markers that can be used to determine the depth of the endoscope in a patient. The clinician identifies a mark relative to a landmark, such as the patient's mouth, prior to removing the endoscope. Upon re-insertion of the endoscope into the patient, the clinician inserts the endoscope until the identified mark is again aligned with the landmark.

Alternately, a depth sensor 515 is connected to the handle of the endoscope and to the entrance of the body where the endoscope is inserted (the bite block or ET tube in the case of a bronchoscope), and returns an electronic signal that indicates the change in distance between the distal tip of the endoscope and the body entrance site. This distance is projected on the image display monitor in order to allow the endoscope operator to see the distance the endoscope is inserted in real time. In addition, a button or other input device is actuated when the tip of the endoscope is at certain locations of interest in the body in order to remember or “bookmark” these locations. These marks can indicate, in the case of a bronchoscope, the location of carinas or branch points in the lung.

When the distal tip of the endoscope is returned to the “bookmarked” position, the display can change to notify the user of proximity to the bookmarked location. In one embodiment, the monitor displays an analog signal, for example a rising bar, which increases in height the closer the tip of the endoscope is to the bookmarked location. The display can also indicate if the bookmarked location is either distal to or proximal to the current location of the tip of the endoscope. This can aid in navigation of the lung and in marking places the operator wishes to return in order to re-observe the tissue, or to perform a procedure such as implanting a device.

Endoscope Tip Location Sensor

At least one company, SuperDimension, has developed a catheter that has a passive receiver in the tip that, when combined with an RF emitter/sensor pad placed under the patient, allows the location and orientation of the tip of the catheter to be determined and superimposed on a 3-dimensional database model of the patient's lungs. In an embodiment, a passive receiver is placed in the tip of an endoscope, thus allowing the location and orientation of the tip of the endoscope to be determined in real time. This allows the user to plan the procedure in advance by examining an imaging scan (such as a CT scan) of the patient taken prior to the procedure. If the goal of the procedure, for example, is to implant bronchial isolation devices in order to isolate a portion of the lungs of a patient, a CT scan of the chest is taken. The intended implant locations for bronchial isolation devices are determined through examination of the scan and indicated in a 3D reconstruction of the CT scan.

During the implant procedure, the intended implant locations are displayed on a monitor visible by the doctor performing the procedure. As the bronchoscope is advanced into the patient's lungs, the bronchoscope transmits an image such as from the tip of the bronchoscope. A visual indication of the intended implant locations (such as a color change) is displayed over the video image taken from the tip of the bronchoscope. In this way, the tip of the bronchoscope can be placed in the preplanned implant location, and the device delivered in the intended location.

Alternately, the position of the scope is displayed in a 3D reconstructed CT scan of the patient's bronchial tree in what is known as a virtual bronchoscopy. The intended implant locations are indicated in the reconstructed image, and as the tip of the bronchoscope progresses through the bronchial tree of the patient, the image of the 3D reconstructed CT scan changes to correspond to the image that would be seen at the tip of the bronchoscope. In this way, the operator can advance the bronchoscope until the tip is located at the intended implant location. The implant procedure can then be performed in the predetermined location.

Flow Control Device Placement

One use of flexible bronchoscopes is to deploy flow control devices, such as one-way valves, into the bronchial lumens of the lung. The following references describe exemplary flow control devices: U.S. Pat. No. 5,954,766 entitled “Body Fluid Flow Control Device”; U.S. Pat. No. 6,694,979, entitled “Methods and Devices for Use in Performing Pulmonary Procedures”; and U.S. Pat. No. 6,941,950, entitled “Bronchial Flow Control Devices and Methods of Use”. The foregoing references are all incorporated by reference in their entirety and are all assigned to Emphasys Medical, Inc., the assignee of the instant application.

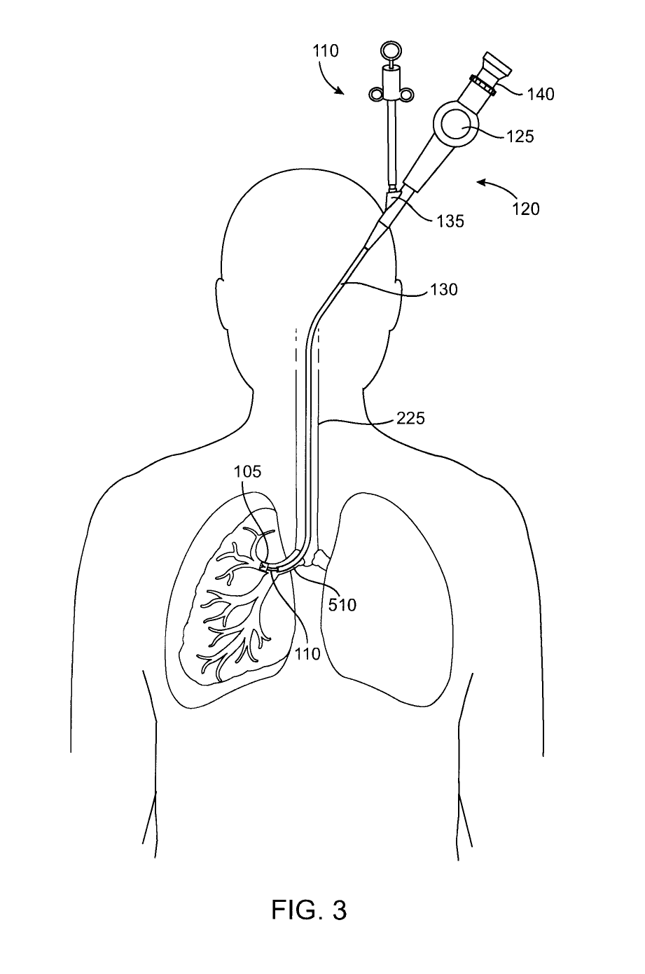

The flow control device is typically deployed from a catheter placed through the working channel of the bronchoscope and into the target bronchial lumen, as shown in FIG. 3. It would be very advantageous to fix the location of the distal tip of the bronchoscope during the delivery of the flow control device in order to improve the accuracy of the placement of the device. This could be accomplished, as shown in FIG. 5, by locating an inflatable balloon 160 around the distal tip of the bronchoscope 120 that could be inflated in order to stabilize and temporarily fix the location of the endoscope tip in the bronchial lumen. The balloon 160 inflates to a size that forms an interference engagement with the inner wall of the lumen to thereby fix the balloon relative to the lumen. Once this is done, a delivery catheter containing the device to be delivered can be advanced through the working channel of the bronchoscope and into the target location. Placement accuracy is improved as the position of the bronchoscope is fixed relative to the bronchial lumen during the implant procedure.

In another embodiment, a self-expanding stent-like cage is mounted to the outside of the distal tip of the bronchoscope. Thus, the balloon 160 shown in FIG. 5 is replaced with an expanding cage. Once the bronchoscope is at a desired location in the lumen, the cage is released and allowed to expand into contact with the inner diameter of the bronchial lumen in order to stabilize and fix the distal tip of the endoscope in place. Of course, this stabilization technique is suitable for the placement of devices other than flow control device, such as stents, into parts of the body other than the lungs, and would also be beneficial during many other endoscopic procedures such as cryotherapy, brachytherapy, etc.

Navigation Improvement

With the advent of therapies that require placing devices or performing therapies in distal anatomy, it is often it is necessary to navigate the endoscope through highly angled and tortuous anatomy. The placement of bronchial isolation devices in the lung, for example, often requires the placement of devices into the segmental and sub-segmental bronchial lumens. If the placement location is one of the upper lobes of the lung, the distal tip of the bronchoscope must articulate to angles near to or exceeding 180 degrees in order to access the segmental or sub-segmental lumens of the upper lobes. The tip of a typical bronchoscope is designed to articulate at the distal tip 180 degrees in one direction, and 130 degrees in the opposite direction when the operator actuates the steering control located on the handle. In order to reach some distal anatomy, 180 degrees of user controlled angulation at the distal tip is not sufficient to allow the tip of the bronchoscope to be inserted into the desired segment or subsegment. It would thus be advantageous to provide methods and devices to improve the amount of articulation at the tip of the endoscope.

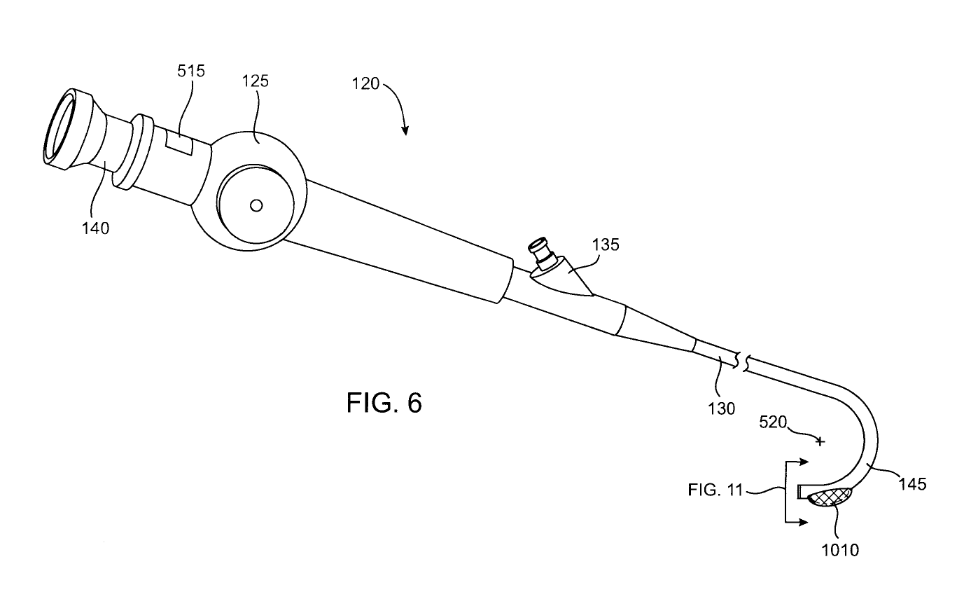

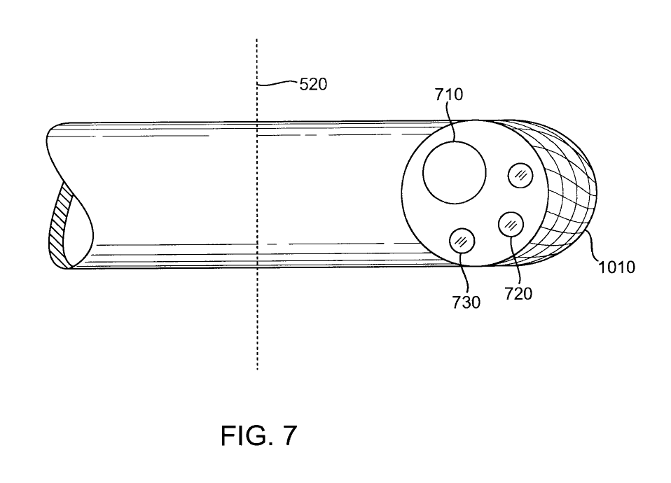

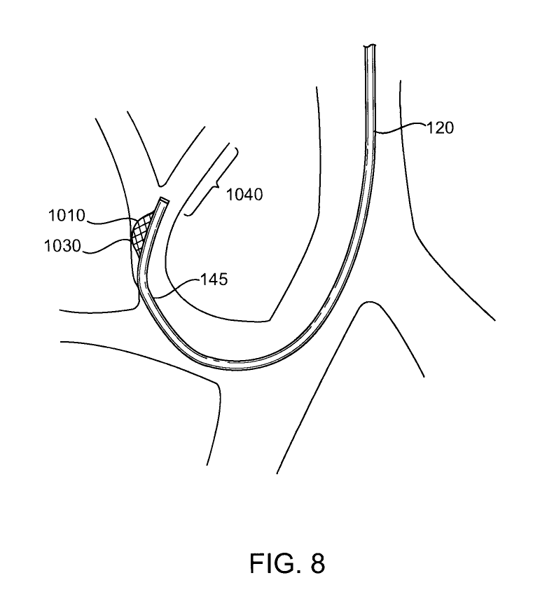

In one embodiment shown in FIG. 6, an inflatable balloon or bladder 1010 is incorporated into the side of the distal tip of the bronchoscope 120. The bronchoscope can include an internal lumen that communicates with the balloon for passing an inflation medium into the balloon for inflating the balloon. The balloon 1010 is located as near to the distal tip 145 of the bronchoscope as possible, and is asymmetrical in that when inflated it expands in only one direction (relative to the longitudinal axis of the bronchoscope) as shown in FIG. 7, unlike the balloon 160 shown in FIG. 5. The balloon is located on the side of the distal tip 145 that is facing radially outwards from the outside edge of the distal tip 145 when it is maximally articulated, and on the side opposite the center of curvature 520 of the distal tip of the endoscope. In this way, when the operator is attempting to access a bronchial lumen that cannot be accessed even with the distal tip 145 maximally articulated, for example to 180 degrees, the balloon 1010 may be inflated to push the distal tip off of the bronchial lumen wall 1030 and into an articulation that is greater than 180 degree as shown in FIG. 8.

In use, the endoscope is inserted into a body lumen of the patient, such as into a bronchial lumen. The distal region of the endoscope is positioned at a location where the balloon can be inflated to push the distal region off of the bronchial wall. The balloon 1010 may then be deflated and the intended procedure performed. Of course, the balloon 1010 may be located at any position around the tip of the endoscope, and more than one balloon may be positioned on the tip of the endoscope. In addition, the balloon may be replaced by any mechanism that would push the distal tip of the endoscope off of an adjacent surface. This could be a lever that is actuated by an electric solenoid, a motor, by hydraulics or by any other method.

In another embodiment, an endoscope having a balloon that expands outwards in all radial directions (rather than on just one side of the endoscope) is inflated to push the distal tip off of the bronchial lumen wall 1030 and into a difficult-to-navigate articulation position. For example, the endoscope shown in FIG. 5 can be used. In such an embodiment, the balloon is inflated such that the balloon increases in diameter to push the distal tip off of the lumen wall. Unlike the previous embodiment, the balloon radially expands outward from all sides of the endoscope, so it is possible that the amount of articulation may be limited with respect to when the balloon pushes from only one side of the endoscope.

Working/Suction/Biopsy Channel Improvements

The working channel of an endoscope is used for many tasks including the insertion of tools such as grasping forceps, the insertion of treatment catheters such as flow control device delivery catheters or cryotherapy catheters, etc. In addition, a suction line may be attached to a port on the endoscope handle, and suction may be applied to the proximal end of the working channel in order to suction secretions and other body fluids through the endoscope and out of the body.

Fluids such as mucus can be left behind in the working channel after incomplete suctioning. Such fluids are later pushed back out of the distal tip of the endoscope when a tool or catheter is inserted through the working channel and out the distal tip. This can result in the fluid blurring or obscuring the field of view, and it would be very beneficial to reduce or eliminate this problem. In one embodiment, the endoscope includes two working channels where one is for the insertion of tools or catheters, and the other is used for suctioning. In this way, secretions or fluids may be left in the suction channel without adverse effects as tools or catheters may be inserted or removed through another working channel. In another embodiment, the working channel of the endoscope has an adjustable aperture at the distal tip of the endoscope that may be adjusted in size by a control at the handle of the endoscope. When suctioning, the aperture diameter may be reduced to increase the air speed of the suction flow through the tip, thus improving the removal of secretions from the working channel.

Another difficulty with existing working channels is that anesthesia gasses can be suctioned out of the lungs of the patient during aggressive suctioning of mucus, and this can cause the patient to become light on anesthesia, or cause the patient to de-saturate due to reduced oxygen supply. One way of reducing this effect is to use a bronchoscope that includes a second, narrow working channel that is connected to a valved gas source, most preferably the same gas source as it used for anesthesia. When the suction valve is opened, the valve on the second working channel is opened and anesthesia gas flows into the patient, preferably at the same rate as gas is removed through the suction channel. Alternately, the second channel can be open to room air, and replacement gas is drawn in passively during suctioning.

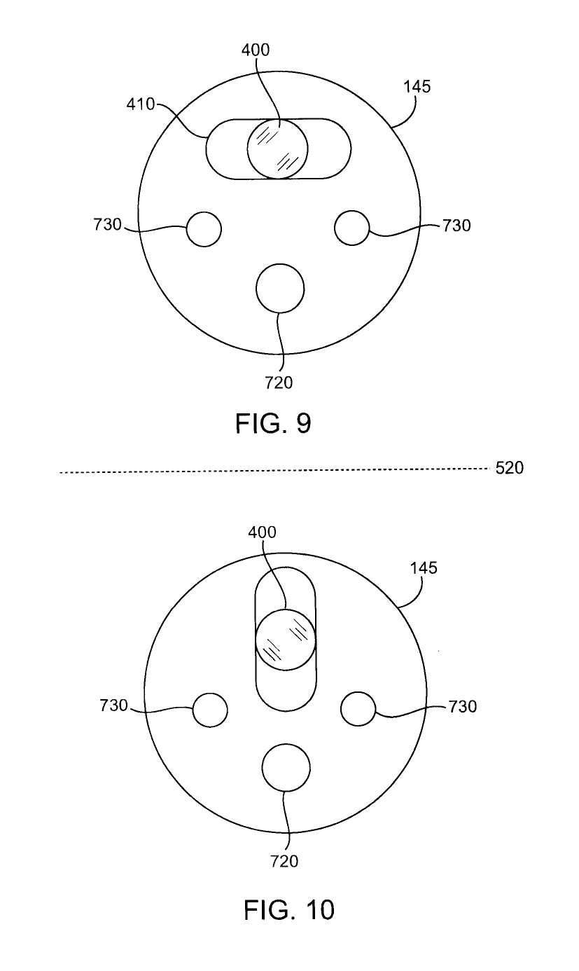

Yet another difficulty with the working channels of existing flexible endoscopes is that in most cases, a tool or catheter placed into the working channel must be removed prior to initializing suctioning as the majority of the working channel is blocked if the tool or catheter is left in place. One way to improve the working channel would be to provide the channel with a shape, such as an oval shape, that allows suctioning around tools and catheters, such as shown in the end view of the distal end of the endoscope illustrated in FIG. 9. A catheter or tool 400 is shown inside the oval working channel 410 which allows suction, and thus fluids, to be pulled around the catheter or tool 400 and through the working channel 410.

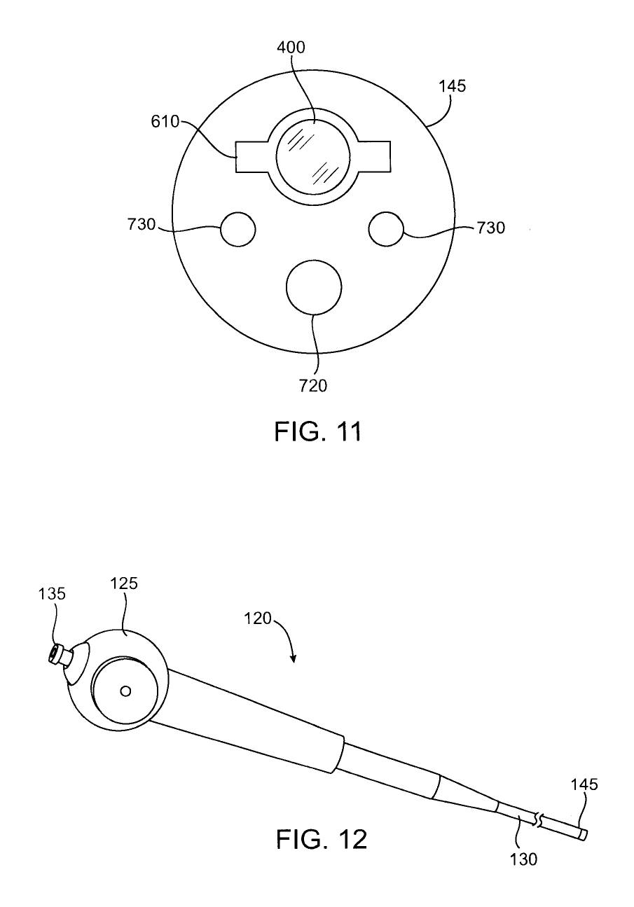

As shown in FIG. 10, if the oval working channel 510 is oriented so that the narrow dimension of the oval is parallel to the center of curvature 520 of the endoscope, it would also be easier to insert tools or catheters 400 into the working channel when the distal end of the endoscope was articulated. Other cross sections of the working channel designed to improve suctioning with a tool or catheter in place are possible. As shown in FIG. 11, the working channel 610 could have a keyhole shape or other shapes that all allow secretions to be suctioned with a tool or catheter in place.

Yet another difficulty with existing flexible endoscopes is that the working channel bends at an angle near the entrance on the handle end of the endoscope, and this requires that tools or catheters inserted in the working channel must bend to conform to this bend in the working channel. This makes it more difficult to insert and remove tools and catheters from the working channel. This design is likely a result of the fact that in first generation flexible endoscopes, the image was transferred from the tip of the endoscope to and eyepiece on the most proximal end of the handle through a coherent fiberoptic bundle. It was necessary with this design to have the working channel bend to allow tools and catheters to be inserted from the side. With the current generation of flexible endoscopes, the image is captured with a CCD camera located at the distal tip of the bronchoscope. Given that there is no eyepiece on the proximal end of the handle, the working channel is configured to run straight through the handle without a bend. As shown in FIG. 12, this permits the working channel entrance 135 to be on the rearmost portion of the handle 125. This makes it much easier to insert and remove tools and catheters through the working channel.

Catheter with Camera

It is often difficult to reach and visualize bronchial anatomy that is deep within the lungs as standard flexible bronchoscopes are either too short, or are too large in diameter. One option is to make the bronchoscope longer and smaller in diameter. One problem with making bronchoscopes smaller in diameter is that the working channel must become smaller as well in order to fit within the smaller sized bronchoscope. This would make the bronchoscope less suitable for use with many pulmonary interventions such as the implantation of bronchial isolation devices.

In an embodiment, a CCD camera (or other type of image collector) is mounted on the tip of a catheter that fits through the working channel of a flexible bronchoscope. For example, the catheter 110 shown in FIG. 3 includes a CCD camera mounted on its distal end. The CCD camera provides an image for visualizing bronchial lumens and other structures deep within the lung. In an embodiment, the camera points straight ahead from the tip of the catheter (along the longitudinal axis or centerline of the catheter). In another embodiment, the camera is positioned to provide an image at a 90 degree angle or at any other angle relative to the centerline of the catheter.

The tip of the catheter can contain a light source or light sources. One or more image transmitting means, such as wires, are located along the length of the catheter from the CCD camera at the distal tip to an image processing unit located outside the patient's body. Alternately, the image can be wirelessly transmitted. The image processor is connected a monitor to allow the image to be viewed by the bronchoscope operator.

In use, if the operator wants to view an anatomical structure that is beyond the reach of the tip of the bronchoscope, the catheter 110 with the CCD camera is inserted into the working channel of the bronchoscope and extended out of the distal tip of the bronchoscope to image the desired anatomy. The catheter 110 can include at least a small amount of controllable tip articulation to allow the catheter tip to be deflected so that it can be guided into angled anatomy.

Although embodiments of various methods and devices are described herein in detail with reference to certain versions, it should be appreciated that other versions, embodiments, methods of use, and combinations thereof are also possible. Therefore the spirit and endoscope of the appended claims should not be limited to the description of the embodiments contained herein.

What is claimed:

1. A delivery system for insertion into a lung, comprising:
a flexible, elongate endoscope extending along a longitudinal axis when the endoscope is straightened;
a first lens located on the distal region, wherein the first lens faces a direction transverse to the longitudinal axis.

2. A system as in claim 1, further comprising a second lens located on a distal region of the endoscope wherein the second lens faces a direction substantially parallel with the longitudinal axis.

3. A system as in claim 1, wherein the first lens faces a direction normal to the longitudinal axis.

4. A system as in claim 2, further comprising a first image collector on a distal region of the endoscope and a second image collector on the distal region of the endoscope.

5. A system as in claim 4, wherein at least one of the image collectors provides an image, and further comprising a processor coupled to the image collector, the processor adapted to digitally alter the image in a manner that flattens the image.

6. A system as in claim 4, wherein at least one of the image collectors provides an image, and further comprising a processor coupled to the image collector, the processor adapted to zoom the image.

7. A delivery system for insertion into a lung, comprising:
a flexible, elongate endoscope extending along a longitudinal axis when the endoscope is straightened;
a depth sensor adapted to be coupled to the endoscope and to a landmark relative to the patient's body, wherein the depth sensor is adapted to provide an electronic signal that indicates the distance between the distal tip of the endoscope and the entrance location.

8. A system as in claim 7, wherein the wherein the depth sensor is further adapted to provide an electronic signal that indicates a change in distance between the distal tip of the endoscope and the entrance location.

9. A system as in claim 7, further comprising a display monitor coupled to the depth sensor, wherein the depth sensor transmits a real-time indication of the distance.

10. A delivery system for insertion into a lung, comprising:
a flexible, elongate endoscope extending along a longitudinal axis when the endoscope is straightened;
a first lens facing a direction normal to the longitudinal axis;
a lumen size measurement device coupled to a distal region of the endoscope, the lumen size measurement device adapted to provide an indication of the size of a bronchial lumen in which the distal region of the endoscope is positioned.

11. A system as in claim 10, wherein the lumen size measurement device comprises an inflatable balloon on a distal region of the endoscope, wherein the inflatable balloon can be inflated to enlarge and contact internal walls of the lumen such that the inflated size of the balloon provides an indication as to the size of the lumen.

12. A system as in claim 10, wherein the lumen size measurement device comprises an ultrasound sensor that provides a cross-sectional density map of the bronchial lumen.

13. A system as in claim 12, wherein the ultrasound sensor is attached to an inflatable balloon on the distal region of the endoscope.

14. A delivery system for insertion into a lung, comprising:
an elongate body having a proximal end and a distal region, the elongate body adapted to be inserted into the lung via a patient's mouth;
a balloon attached to the distal region, the balloon adapted to be inflated in an asymmetrical manner such that the balloon expands in only one direction relative to a longitudinal axis of elongate body.

15. A delivery system as in claim 14, wherein the balloon is attached to a distal end of the elongate body.

16. A delivery system as in claim 14, wherein the balloon expands in a direction that is opposite a center of curvature of the elongate body.

17. A delivery system for insertion into a lung, comprising:
a flexible endoscope adapted to be inserted into the lung;
an orientation indicator coupled to the flexible endoscope, the orientation indicator adapted to provide an indication as to the direction to an anatomical structure of a patient in which the endoscope is inserted regardless of the orientation of the endoscope within the patient.

18. A delivery system as in claim 17, wherein the orientation indicator includes a display monitor that provides the indication.

19. A delivery system as in claim 18, wherein the indication is an arrow that is displayed on the display monitor.

20. A delivery system as in claim 17, wherein the orientation indicator includes a rotation sensor disposed on the endoscope wherein the rotation sensor provides an indication of a change in angle that the endoscope is rotated relative to device fixed relative to the patient.

21. A delivery system as in claim 17, wherein the orientation indicator includes a directional radio frequency (RF) sensor disposed on the endoscope and an RF emitter pad that is fixed relative to the patient.
